(19) United States
(12) Patent Application Publication
(10) Pub. No.: US 2024/0400599 A1
(43) Pub. Date: Dec. 5, 2024

(54) LIGNIN, METHODS OF EXTRACTION AND USES THEREOF

(71) Applicants: UNIVERSIDADE CATÓLICA POR TUGUESA - UCP, Porto (PT); AMYRIS BIO PRODUC TS PORTUGAL UNIPESSOAL LDA, Porto (PT)

(72) Inventors: Patrícia Filipa SANTOS COSTA, Porto (PT); Maria Inês FERREIRA DA MOTA, Porto (PT); Filipa Alexandre SOARES GOMES ANTUNES, Porto (PT); João Porfírio DA SILVA BURGAL, Porto (PT); Maria Manuela ESTEVEZ PINTADO, Porto (PT)

(73) Assignees: UNIVERSIDADE CATÓLICA PORTUGUESA - UCP, Porto (PT); AMYRIS BIO PRODUCTS PORTUGAL UNIPESSOAL LDA, Porto (PT)

(21) Appl. No.: 18/700,893

(22) PCT Filed: Oct. 12, 2022
(86) PCT No.: PCT/IB2022/059789

§ 371 Date: Apr. 12, 2024

(30) Foreign Application Priority Data
Oct. 12, 2021 (PT) 117515

(51) Int. Cl. C07G 1/00; A61K 8/72; A61Q 17/04; A61Q 19/08

(52) U.S. Cl. CPC C07G 1/00; A61K 8/72; A61Q 17/04; A61Q 19/08; A61K 2800/51; A61K 2800/524

(57) ABSTRACT

The present disclosure relates to a light-brown lignin, with a sweet and woody odour, antioxidant activity and ultraviolet protection for use in cosmetics. The present disclosure also relates to an alkaline method of extracting said lignin from sugarcane bagasse using mild conditions, the lignin obtained by the method of the present disclosure also has a high purity. The lignin of the present disclosure may be used in cosmetic composition, such us beauty products, in particular a face cream, more in particular blemish balm (BB) cream.

### TECHNICAL FIELD

The present disclosure relates to a light-brown lignin, preferably lignin, with a sweet and woody odour, antioxidant activity, and ultraviolet protection for use in cosmetics. The present disclosure also relates to an alkaline method of extracting said lignin from sugarcane bagasse (SCB) using mild conditions, the lignin obtained by the method of the present disclosure also has a high purity. The lignin of the present disclosure may be used in cosmetic composition, such us beauty products, in particular a face cream, further in particular a blemish balm (BB) cream.

### BACKGROUND

Recent concerns about climatic change and the exploration of alternatives to fossil fuels has focused global attention on sugarcane as a source of biomass. The annual global production of sugarcane is about 328 Mt being Asia the main production region (44%) followed by South America (34%) (Sindhu et al. 2016). The significance of the sugarcane industry is not only due to sugar production but also to its by-products. Sugar production from sugarcane generates several by-products that can be used for energy production. Although highly appealing for environmental and financial reasons, it still remains economically unattractive. In this context, the conversion of by-products into value added compounds and applications is crucial. The main solid by-products include plant tops, straw, bagasse, filter cake and molasses, which can be grouped into two stages: those originated during the harvesting stage (tops and straw), and those produced during industrial processing (bagasse, filter cake, and molasses). The main components of the solid by-products include cellulose, hemicellulose and lignin.

Sugarcane is a large perennial tropical grass belonging to the family Gramineae and the genus Saccharum officinarum. Sugarcane is a major crop cultivated globally for sugar production with relevant features as high biomass yield, high sucrose content and high efficiency in accumulating solar energy. After the harvest of sugarcane, the sugarcane stalks are processed in sugar mills for the extraction of cane juice, while the leaves and tops are left in the cane field. Two major by-products from the sugarcane industry are the harvest residue (straw) and the fibrous fraction following juice extraction (bagasse). These post-harvest by-products have been suggested as an abundant and inexpensive source of lignocellulosic biomass. Sugarcane bagasse (SCB) and sugarcane straw (SCS) are basically composed of cellulose, hemicellulose, and lignin, with lower amounts of extractives and ash. SCB is almost completely used by the sugar industry as fuel for the boilers, while SCS is commonly used as animal fodder or burnt in the field (Sindhu et al. 2016). Lignocellulosic biomass has been recognized for its potential use to produce chemicals and materials, having the advantages of low cost and availability.

Lignin is the second most abundant biopolymer in nature. The main functions attributed to lignin in the plant are elasticity and mechanical strength. It is a complex aromatic macromolecule formed by the dehydrogenative polymerization of three phenylpropanoid monomers coniferyl, synapyl and p-coumaryl alcohols. In the specific case of sugarcane lignin, it is greatly acylated (p-coumaroylation) at their side chains, contain tricin flavonoid units and have ferulate residues cross-coupled between arabinoxylan and lignin (del Río et al. 2015).

The production of high-value lignin-derived products is still a challenge due to the complex structure of lignin, polydispersity, recalcitrant nature, dependence on the type of biomass, amongst others. Additionally, lignin isolation, fractionation, modification, and characterization remain a challenge. Usually, the pre-treatment process drives the separation of the lignocellulosic biomass into the main components as an efficient way of reducing natural recalcitrance of the lignocellulose cell wall (Liao et al. 2020). A suitable pre-treatment method aims to efficiently extract lignin from the lignocellulosic and generate a lignin fraction of high purity and quality that can achieve the requirements for subsequent conversion steps.

Lignin is part of the cellular wall and confers structural support, hydrophobicity and resistance against microbial attack and oxidative stress, and among the components of lignocellulose, it is the most recalcitrant to chemical and biological degradation. Hemicellulose is linked to cellulose and lignin by covalent bonds and fewer hydrogen bonds. Lignin acts like a glue and bind cellulose and hemicellulose, which in turn makes the structure more moisture resistant and recalcitrant to chemical and biological degradation. Researches have been conducted in exploring isolation methods and potential applications. These wastes streams, SCB and SCS, may constitute a lignocellulosic source in countries with high sugarcane production such as Brazil, India and China (Sindhu et al. 2016). Both SCB and SCS have a high cellulose (30-40 wt. %) content, which could be used to produce Second Generation (2G) ethanol via different chemical, physical or biological pre-treatments to convert them into fermentable sugars, while separating and valorising lignin as well. Another positive aspect of sugarcane is that it is not required an increase in the harvesting area because this residue has a high regeneration capacity and yield (80 t/ha), thus not competing with arable land.

There is literature devoted to sugarcane bagasse deconstruction by hydrothermal, alkaline, acidic or organosolv pre-treatments and to different applications of the respective fractions, ranging from fuels (bioethanol), bioactive extractives (lipophilic or phenolic-rich extracts), high added value molecules and biomaterials. Lignin can be extracted using several methods such as alkaline, which include kraft and soda pulping, and aqueous alkaline pre-treatment; acidic, with organosolv and steam explosion pre-treatment and extraction; reductive catalytic fractionation; ionic liquid dissolution; and mechanical pre-treatment. After extracting lignin from the biomass, it is necessary to perform a separation and isolation step. Currently, the biggest supplier of lignin is the paper and pulp industry (in the form of black liquors), which utilizes an alkaline process (kraft process) (Arni 2018; Liao et al. 2020).

Traditionally, lignin is obtained from black liquor by precipitation methods, involving the use of acids, mostly with sulphuric acid, and more recently with a combination of carbon dioxide and sulfuric acid. At approximately pH 4, complete lignin precipitation has been observed by most researchers. It is well known that the ionization of phenolic groups plays a major role in the solubility of kraft lignin at alkaline pH. The apparent pKa value of lignin is a function of several parameters such as the chemical substitution pattern on the phenolic aromatic ring, temperature and solution conditions (Sewring et al. 2019).

The structural characteristics of lignin depend on several factors including the botanical origin, environmental growth, and extraction conditions. A study with lignin from SCB extracted with different chemical procedures using ethanol and alkaline solutions was performed to evaluate their potential as antioxidant. Antioxidant activity of alkaline lignin was stronger than ethanol lignin due to its higher quantities of phenolic hydroxyl and methoxy groups that influenced more than its molecular mass and polydispersity (Li and Ge 2012).

Solar ultraviolet (UV) radiation is a causative factor of polymer and pigments degradation (Yousif and Haddad 2013). Several studies have shown the potential of lignin to prevent materials damage by blocking UV radiation (Sirvio et al. 2020). This property is associated with the ability of the phenolic groups to trigger radical scavenging. These products are categorized according to their active ingredients: physical or chemical sunscreens. In physical sunscreens, the active ingredients are mainly zinc oxide and titanium dioxide, based on the reflection mechanism. On the other hand, chemical sunscreens act via an UV radiation absorbing mechanism. Comparing both physical and chemicals UV blockers, the chemical ones do offer certain advantages, such as greater ease of application onto the skin, as well as greater comfort. However, the synthetic chemicals may have negative effects on skin tissue. Hence, there has been increasing attention placed on the use of natural compounds in sunblock applications, due to their good UV radiation protection properties and antioxidant activities. Many natural polyphenol extracts have poor photo-stability. Therefore, more stable natural macromolecular sunscreens are needed. Lignin has a complex three-dimensional structure containing high carbon content, UV chromophore groups, and aromatic rings of hydroxyl and methoxy groups along with double bonds and carbonyl functional groups. These properties make it a good candidate as a natural UV blocker agent (Widsten 2020).

Lignin is an attractive biopolymer due to its availability in nature, biodegradability and UV-blocking properties. The chemical structure of lignin allows a variety of modifications that turns it into a potential building block for biopolymer synthesis, blends, and biocomposites. For example, the antioxidant activity of lignin plays an important role in the design of products. This property is associated with the ability of the phenolic groups to trigger radical scavenging.

The typical dark colour of lignin is also a known constrain that limits its use in several applications and affects the final colour of the product. Since the early 1980s, research efforts have been made to understand the main factors responsible for attributing colour to lignin and/or develop methodologies to reduce its colour. It is assumed that coloured groups arise from chromophores and leucochromophores formation coming from lignin and carbohydrates. Lignin-based chromophores contain carbonyl functional groups, conjugated phenolics, quinoid structures and metal complexes. Some chromophores and leucochromophores originated from lignin include several quinones, catechol, among others; however, it is not possible yet to identify which ones are present in lignin samples.

Research studies indicate that besides the biomass source, unit operations involved in lignin production such as delignification, precipitation, recovery, and fractionation processes also have impact on colour since they are responsible for the formation or elimination of multiple-bond functional groups. Milder delignification conditions and lignin re-slurrying in acidic water usually result in brighter lignins. Suggested methodologies to reduce lignin colour submit lignin to chemical (e.g. oxidation or solvent fractionation) or biological (e.g. with fungi) processes.

Nevertheless, these suggested processes are not yet cost-effective.

Typical strong lignin odour is usually attributed to small molecules originated from lignin itself (e.g. guaiacol) or delignification process (e.g. dimethyl disulfide) (Guggenberger et al. 2019). Guaiacol is one of the low-molecular weight compounds responsible for the typical smoky and woody odour of lignins.

These facts are disclosed in order to illustrate the technical problem addressed by the present disclosure.

### REFERENCES

1) Arni, Saleh Al. 2018. “Extraction and Isolation Methods for Lignin Separation from Sugarcane Bagasse: A Review.” Industrial Crops and Products 115:330-39.
2) Canilha, Larissa, Victor T. O. Santos, George J. M. Rocha, João B. Almeida e Silva, Marco Giulietti, Silvio S. Silva, Maria G. A. Felipe, Andre Ferraz, Adriane M. F. Milagres, and Walter Carvalho. 2011. “A Study on the Pretreatment of a Sugarcane Bagasse Sample with Dilute Sulfuric Acid.” Journal of Industrial Microbiology & Biotechnology 38(9):1467-75.
3) da Silva, Ayla Sant'Ana, Hiroyuki Inoue, Takashi Endo, Shinichi Yano, and Elba P. S. Bon. 2010. “Milling Pretreatment of Sugarcane Bagasse and Straw for Enzymatic Hydrolysis and Ethanol Fermentation.” Bioresource Technology 101(19):7402-9.
4) del Río, José C., Alessandro G. Lino, Jorge L. Colodette, Claudio F. Lima, Ana Gutiérrez, Ángel T. Martínez, Fachuang Lu, John Ralph, and Jorge Rencoret. 2015. “Differences in the Chemical Structure of the Lignins from Sugarcane Bagasse and Straw.” Biomass and Bioenergy 81:322-38.
5) Guggenberger, Matthias, Antje Potthast, Thomas Rosenau, and Stefan Böhmdorfer. 2019. “Quantification of Volatiles from Technical Lignins by Multiple Headspace Sampling-Solid-Phase Microextraction-Gas Chromatography-Mass Spectrometry.” ACS Sustainable Chemistry & Engineering 7(11):9896-9903.
6) Li, Zhili and Yuanyuan Ge. 2012. “Antioxidant Activities of Lignin Extracted from Sugarcane Bagasse via Different Chemical Procedures.” International Journal of Biological Macromolecules 51(5):1116-20.
7) Liao, Jing Jing, Nur Hanis Abd Latif, Djalal Trache, Nicolas Brosse, and M. Hazwan Hussin. 2020. “Current Advancement on the Isolation, Characterization and Application of Lignin.” International Journal of Biological Macromolecules 162:985-1024.
8) Rabelo, S. C., H. Carrere, R. Maciel Filho, and A. C. Costa. 2011. “Production of Bioethanol, Methane and Heat from Sugarcane Bagasse in a Biorefinery Concept.” Bioresource Technology 102(17):7887-95.
9) Rocha, G. J. M., A. R. Gonçalves, B. R. Oliveira, E. G. Olivares, and C. E. V. Rossell. 2012. “Steam Explosion Pretreatment Reproduction and Alkaline Delignification Reactions Performed on a Pilot Scale with Sugarcane Bagasse for Bioethanol Production.” Industrial Crops and Products 35(1):274-79.
10) Sewring, Tor, Julie Durruty, Lynn Schneider, Helen Schneider, Tuve Mattsson, and Hans Theliander. 2019. “Acid Precipitation of Kraft Lignin from Aqueous Solutions: The Influence of PH, Temperature, and Xylan.” Journal of Wood Chemistry and Technology.
11) Sindhu, Raveendran, Edgard Gnansounou, Parameswaran Binod, and Ashok Pandey. 2016. “Bioconversion of Sugarcane Crop Residue for Value Added Products—An Overview.” Renewable Energy 98:203-15.
12) Sirviö, Juho Antti, Mostafa Y. Ismail, Kaitao Zhang, Mysore V. Tejesvi, and Ari Ämmälä. 2020. “Transparent Lignin-Containing Wood Nanofiber Films with UV-Blocking, Oxygen Barrier, and Anti-Microbial Properties.” Journal of Materials Chemistry A 8(16):7935-46.
13) Widsten, Petri. 2020. “Lignin-Based Sunscreens—State-of-the-Art, Prospects and Challenges.”
14) Yousif, Emad and Raghad Haddad. 2013. “Photodegradation and Photostabilization of Polymers, Especially Polystyrene: Review.” SpringerPlus 2(1):398.

### GENERAL DESCRIPTION

The present disclosure relates to a light-brown lignin, with sweet odour with notes of paper wood and wooden pencil, improve antioxidant activity and ultraviolet protection activity for use in cosmetics. The present disclosure also relates to an alkaline method of extracting said lignin from sugarcane bagasse (SCB) using mild conditions, the lignin obtained by the method of the present disclosure also has a high purity.

An aspect of the present disclosure relates to cosmetics comprising light-brown lignin, with sweet odour with notes of paper wood and wooden pencil, antioxidant activity and ultraviolet protection, wherein the lignin is obtained using the method of the present disclosure.

The method of the present disclosure produces a lignin with sweet odour with notes of paper wood and wooden pencil and lighter colour to be obtained without the need for extra lignin-modification steps.

The typical dark colour of lignin is a challenge for its application such as in cosmetics. Biomass fractionation, lignin extraction and recovery involve formation and/or elimination of multiple-bond functional groups. In order to lighten colour of lignin, different solutions have been proposed including, for example, lignin fractionation using methanol/water solvent, irradiating lignin by UV irradiation in tetrahydrofuran solution, or by blocking the free phenolic hydroxyl of lignin and then self-assembling into colloidal spheres. Lignin is also known to be UV absorbing compound.

In view of the drawbacks to the prior art, the technical problem underlying the invention was to develop a new method to produce lignin with a light-brown colour and a pleasant odour, antioxidant and UV blocker activities.

An aspect of the present disclosure relates to a lignin obtained by the method of the present disclosure comprising and wherein lignin has a sweet and woody odour, preferably with notes of paper wood and wooden pencil. In the state of the art, the lignin odour sensorial analysis was performed with a trained descriptive sensory panel of six judges.

The lignin obtained in the present disclosure has the advantage of being light-brown coloured. The lignin dried using spray dryer is lighter brown coloured as compared to the one dried using oven.

An aspect of the present disclosure relates to a lignin comprising a light-brown colour and with a sweet and woody odour, preferably with notes of paper wood and wooden pencil, wherein the colour is at least to L*=45, a*=6, b*=16 scales, measured by the CIELAB system and a particle size inferior to 70 μm; preferably a particle size inferior to 60 μm, wherein said lignin has a sweet odour with notes of paper wood and wooden pencil.

In the state of the art, the colour may be measured by many methods, in the present disclosure the colour was measured by the CIELAB system (or CIE L*a*b*).

In an embodiment, the lignin colour is at least to L*=60 measured by the CIELAB system, more preferably wherein the colour is at least to L*=70 measured by the CIELAB system. These values are achieved without any further bleaching step.

Surprisingly guaiacol is absent in the odour profile of the lignin of the present disclosure. This absence in combination with other factors may explain the woody odour, preferably with notes of paper wood and wooden pencil of the lignin of the present disclosure.

In an embodiment, these values are surprisingly obtained from residues from agriculture, better results were obtained using sugarcane bagasse.

In an embodiment for better results, the lignin is obtained by spray dryer.

In an embodiment, in the lignin of the present disclosure guaiacol is absent (one of volatile organic compounds responsible for the strong smoky and woody odour of lignin). As shown in the FIGS. 5 and 6 the presence of guaiacol was not detected by HS-SPME-GC-MS for lignins of the present disclosure. Surprisingly, the lignin of the present disclosure has a light-brown colour, sweet and woody odour, preferably with notes of paper wood and wooden pencil, UV protection and antioxidant activity.

In the state of the art, the particle size may be measured by many methods, in the present disclosure the particle size was measured was measured by laser diffraction, preferably using the equipment Mastersizer™ 3000.

In an embodiment, the lignin colour is at least to L*=60 measured by the CIELAB system, more preferably wherein the colour is at least to L*=70 measured by the CIELAB system. These values are achieved without any further bleaching step.

In an embodiment, the colour is at least L*=60.3, a*=6.1, b*=19.1 for a lignin obtained by spray dried or L*=48.4, a*=10.0, b*=20.3 scales for a lignin obtained by oven dried.

In the present disclosure, D10 (Dv (10)), D50 (Dv (50)), and D90 (Dv (90)), are called percentile values. These are statistical parameters that can be read directly from the cumulative particle size distribution. They indicate the size below which 10%, 50% or 90% of all particles are found.

In an embodiment, the lignin particle size ranges from 0.5-50 μm, preferably measured by laser diffraction, more preferably the lignin particle size ranges from 1-40 μm.

In the state of the art, the particle size may be measured by many methods, in the present disclosure the particle size was measured was measured by laser diffraction, preferably using the equipment Mastersizer™ 3000.

In an embodiment, the lignin functional groups may be selected from a list consisting of: carboxylic groups (ex. 1.08-1.23 (mmol/g)), free phenolic hydroxyl groups (ex. 1.53-1.77 (mmol/g)).

In an embodiment, the lignin of the present disclosure may be incorporated into a Blemish balm (BB) cream formulation display ultraviolet light absorbance activity ranges from 2-12, measured at the global solar irradiance in the UV wavelength range of 290-400 nm; preferably measured with a spectrophotometer SPF-290AS (Solarlight®, United States of America).

A BB cream is usually defined as a cosmetic product with a creamy formulation, typically lighter in texture than foundation, used to even out facial skin tone.

In an embodiment, the lignin may comprise an antioxidant activity between 0.1-0.5 (mg/ml), preferably 0.2-0.3 (mg/ml), more preferably 0.20-0.27 (mg/ml).

In an embodiment, the lignin may be use in medicine, preferably for prevent solar skin diseases.

In an embodiment, the lignin may be use as UV blocker agent.

The method of the present disclosure allows obtained an improved lignin with a sweet and woody odour, preferably with notes of paper wood and wooden pencil and light colour are obtained in a single extraction process and a high purity. Surprisingly, the degree of purity of the obtained lignin is at least 90%, preferably more than 98%; more preferably more than 99%.

The lignin obtained in the present disclosure has the advantage of having a sweet and woody odour, preferably with notes of paper wood and wooden pencil without any need for further additional steps in the process.

In an embodiment, in order to optimize lignin removal (i.e. delignification yield) from SCB, mild extracting temperature of 90° C. and a liquid to solid ratio of 15 was used and the influence of extracting time and sodium hydroxide concentration were optimized.

In an embodiment, operating conditions for lignin removal is preferably 2-6% (w/v) sodium hydroxide concentration at 90° C. for 0.5-2 hours.

In an embodiment, the particle size of the lignin for use as a UV blocker in cosmetics preferably contains 90% of particles size below 57 μm D(v,0.9). As an attempt aiming at proving the potential of lignin as a colorant and UV blocker, a BB cream containing oven-dried lignin—with heterogeneous particle size—in its composition was formulated. However, problems with lignin dispersion were noticed. To overcome this, lignin was milled using a ball mill and sieved to separate by particle size. A good lignin dispersion in cream formulation was accomplished for sieved particles passing through a sieve of mesh size 40 μm, where 90% of particles are below 38.1 μm (D(v,0.9) (measured by Mastersizer 3000). This fraction was then tested in formulation and was observed to disperse well.

In an embodiment, the spray dryer technique was performed to obtain high concentration of lignin, where 90% of particles size are below 57 μm (Dv (90)) (measured by Mastersizer™ 3000). The fraction below 40 μm (sieve measurement) represented only approximately 14% of all produced lignin obtained via the oven drying technique. The spray-dryer allowed to produce higher amounts of lignin with particle size suitable for cosmetic applications.

In an embodiment, the present disclosure relates to a composition comprising the lignin of the present disclosure and a suitable base.

Another aspect of the present disclosure is the use of the lignin/composition of the present disclosure as a cosmetic additive, as a pigment, as an ultraviolet blocking agent or as an antioxidant.

In an embodiment, the lignin/composition of the present disclosure may be use as an antioxidant and an ultraviolet blocker, namely an ultraviolet blocker booster.

In an embodiment, the lignin/composition of the present disclosure may be use for preventing solar skin diseases.

Another aspect of the present disclosure relates to a composition, preferably an aqueous composition, comprising the lignin described in any of the previous claims and a suitable cosmetic base.

In an embodiment, the suitable cosmetic base is selected from a list consisting of emulsifier, humectant, thickener, stabilizer, preservative, chelating agent, emollient, pH adjuster, or mixtures thereof.

In an embodiment, the emulsifier is selected from a list consisting of: Polyglyceryl-6 distearate, glyceryl stearate, cetearyl alcohol, polysorbate-20, or bee wax.

In an embodiment, the humectant is selected from a list consisting of: Glycerine, propylene glycol, xanthan gum, salicylic acid, or hyaluronic acid, or mixtures thereof.

In an embodiment, the thickener is selected from a list consisting of: Xanthan gum, hydroxyethylcellulose, sclerotium gum, Acacia Senegal gum, carbomers, mixtures thereof.

In an embodiment, the stabilizer is selected from a list consisting of: Acacia Senegal gum, xanthan gum, carbomers, sodium gluconate, triethanolamine, or mixtures thereof.

In an embodiment, the preservative is selected from a list consisting of: Phenethyl alcohol, benzoic acid, sorbic acid, salicylic acid alcohol, pentylene glycol, or mixtures thereof, or mixtures thereof.

In an embodiment, the chelating agent is selected from a list consisting of: Sodium phytate, citric acid, EDTA, sodium oxalate, or mixtures thereof.

In an embodiment, the emollient is selected from a list consisting of: Butyrospermum parkii Butter, caprylic/capric Triglyceride, bee wax, squalane, cetyl alcohol, dimethicone, lanolin, or mixtures thereof.

In an embodiment, the pH adjuster is selected from a list consisting of: Citric acid, aqua/sodium hydroxide, ascorbic acid, acetic acid, magnesium hydroxide, or mixtures thereof.

In an embodiment, the composition comprises 1-10 wt % of lignin and 90-99 wt % of base; preferably 3-7 wt % of lignin and 93-97 wt % of base; more preferably 4-5 wt % of lignin and 95-96 wt % of base.

In an embodiment, the composition is a cosmetic composition, preferably, cosmetic composition for use in preventing solar skin diseases.

In an embodiment, the composition is for use in medicine, therapy or treatment, preferably for preventing solar skin diseases.

In an embodiment, the cosmetic composition is in the form of a topical composition.

In an embodiment, the topical composition is a gel, a lotion, a liquid, a balm, a cream, a serum, a spray, or combinations thereof.

In an embodiment, the topical composition is a blemish balm, colour correcting cream. A blemish balm is commonly known as BB cream and a colour correcting cream is commonly known as CC cream.

In an embodiment, the present disclosure relates to the use of the lignin/composition of the present disclosure as a cosmetic additive, as a pigment, as an ultraviolet blocking agent or as an antioxidant.

In an embodiment, the present disclosure relates to the lignin/composition present disclosure for preventing solar skin diseases.

In an embodiment, the present disclosure relates to a method of preparing the alkaline lignin of the present disclosure from a SCB comprising the step of:
submitting SCB to an alkaline pre-treatment, preferably to a soda (sodium hydroxide) pre-treatment, in order to obtain a black liquor;
acid precipitation of the black liquor obtained in the previous step and collection of the precipitate to obtain lignin;
drying the collected precipitate and optionally milling the drying lignin.

In an embodiment, in order to optimize lignin removal (i.e. delignification yield) from SCB, mild extracting temperature of between 80-120° C., preferably 90° C. and a liquid to solid ratio (v/w) of 12-18 was used. The influence of extracting time (0.5 to 2 hours) and sodium hydroxide concentration (2-6 wt %) were also optimized. Preferably, operating conditions for lignin removal is between 2 wt % sodium hydroxide concentration, at 90° C. for 0.5 hours.

In an embodiment, the precipitation agent is H₂SO₄ 98%, wherein the amount of H₂SO₄ ranges from 10-50% (v/w), preferably from 20-35% (v/w), more preferably 30% (v/w).

In an embodiment, precipitation is performed at room temperature, from 18-25° C., preferably from 20-22° C.

In an embodiment, the time of the precipitation stage ranges from 2-20 minutes, preferably from 5-10 minutes.

In an embodiment, in the precipitation stage, the H₂SO₄ is added in a flow rate ranging from 20-350 mL/min, preferably from 100-260 mL/min.

The precipitation medium reaches a final temperature of 80-95° C.

In an embodiment, the method may further comprise a filtration or centrifugation step.

In an embodiment, the drying is a spray drying or oven drying.

In an embodiment, the degree of purity of the obtained alkaline lignin is at least 85-90%, preferably more than 95%; more preferably more than 99%.

Another aspect of the present is the lignin obtained from the method of the present disclosure, a light-brown colour lignin from sugarcane bagasse, wherein the colour is at least L*=60.3, a*=6.1, b*=19.1 scale, measured by the CIELAB system, wherein guaiacol is absent in the lignin odour profile and wherein lignin has a sweet and woody odour. This lignin has better results when the alkaline pre-treatment is used, and the lignin is from SCB.

### BRIEF DESCRIPTION OF THE DRAWINGS

The following figures provide preferred embodiments for illustrating the disclosure and should not be seen as limiting the scope of invention.

FIG. 1 shows an embodiment of the process of extracting lignin from lignin-rich sugarcane bagasse sample using oven.

FIG. 2 shows an embodiment of the process of extracting lignin from lignin-rich sugarcane bagasse sample using spray drying.

FIG. 3 shows photos of lignin-rich sample dried using an oven (FIG. 3A) and a spray dryer (FIG. 3B).

FIG. 4 shows the particle size distribution of the lignin.

FIG. 5 shows the HS-SPME-GC-MS chromatograms obtained in full scan for A) glycerol (solvent used to disperse lignins for analysis), B) commercial kraft lignin (CAS 8068-05-1), C) lignin produced herein, D) guaiacol standard and E) air (blank sample). HS-SPME-GC-MS was performed by a Bruker 456-GC gas chromatography equipped with a triple quadrupole mass spectral detector (mass range 33-450 m/z). Chromatographic separation was performed employing BR-5 MS column from Restek (USA, length 30 m, inner diameter 0.25 mm and film thickness 25 μm). Injector, transfer line and EI temperatures at 240° C., 280° C. and 230° C., respectively. Column temperature program: 40° C., 1 minutes; 5° C./minutes up to 250° C., hold 5 minutes; 5° C./min up to 300° C. (58 minutes). It was employed a vdivinylbenzene/carboxen/polydimethylsiloxane (DVB/CAR/PDMS)·50/30 mm fibre from Supelco (Bellefonte, PA, USA). The incubation was performed at 40° C. for 10 minutes (500 rpm), the extraction at 40° C. during 30 minutes (500 rpm) and desorption during 10 minutes. SPME penetration depth of 16 mm.

FIG. 6 shows the HS-SPME-GC-MS extracted ion chromatograms at m/z 81 (for targeting guaiacol) obtained for A) glycerol (solvent used to disperse lignins for analysis), B) commercial kraft lignin, C) Lignin produced herein, D) guaiacol standard and E) air (blank sample). Analysis conditions are the same as described in FIG. 5. No guaiacol (retention time: 11.8 minutes) was detected in vials with air, glycerol and Spray dried lignin/glycerol).

FIG. 7 shows example of the MPF values obtained for each performed scan of PMMA (reference sunscreen)

FIG. 8 shows example of the MPF values obtained for each performed scan of PMMA (BB Cream).

### DETAILED DESCRIPTION

The present disclosure relates to a light-brown lignin, with a sweet odour with notes of paper wood and wooden pencil, antioxidant activity, and ultraviolet protection for use in cosmetics. The present disclosure also relates to an alkaline method of extracting said lignin from sugarcane bagasse (SCB) using mild conditions.

Sugarcane composition can vary according to its origin and season of the year; however, it is possible to observe from data collected that the biomass received so far has a very homogeneous composition with similar cellulose content between 39 wt % and 43 wt % with slight variation in the content of hemicellulose of between 19 wt % to 29 wt %, lignin content of between 21 wt % to 27 wt % and inorganics content of between 1 wt % to 4%. The data obtained is in accordance with that reported in literature: 39-45 wt % of cellulose, 23-27 wt % of hemicellulose, 19-32 wt % of lignin, 5-7 wt % of extractives and 1-3 wt % inorganics (Canilha et al. 2011; Rabelo et al. 2011; Rocha et al. 2012; da Silva et al. 2010).

The reagents used in the extraction of lignin from SCB are displayed in Table 1 below.

TABLE 1
CAS number and purity of each reagent.

| | CAS | |
|---|---|---|
| Reagent | Number | Purity |
| Sodium hydroxide (NaOH) | 1310-73-2 | 100% |
| Sulfuric acid (H₂SO₄) | 7664-93-9 | 95-98% |

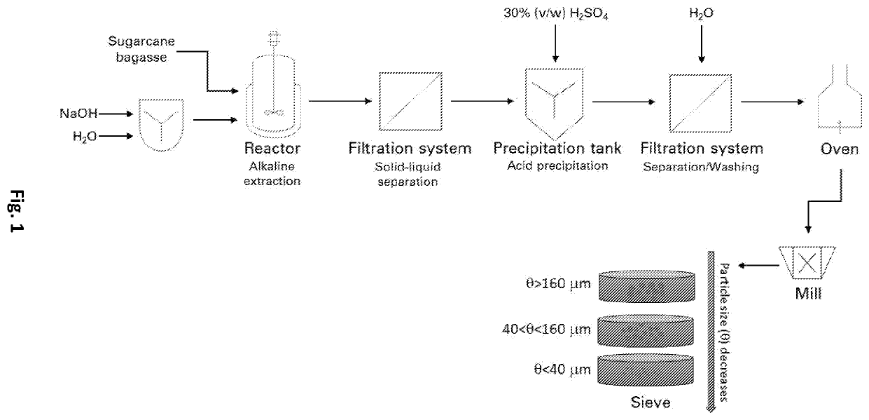

In an embodiment, lignin was extracted from SCB and oven dried. The extraction process of lignin from SCB is as shown in FIG. 1. A brief description of the operating units is as follows:
STAGE 1:
Prepare alkaline solution by adding 2 wt % sodium hydroxide solution to deionized water in a mixer.
Delignification of SCB in a Parr reactor using 2 wt % sodium hydroxide solution in deionized water.
STAGE 2:

Filtering out the extracted SCB biomass (solid) from the liquid stream containing the solubilized lignin (black liquor).
STAGE 3:
Precipitating out the lignin by gradual addition of 98% (v/v) sulfuric acid to the black liquor with stirring (200-500 rpm).
STAGE 4:

Filtering out the lignin solids from the acidified black liquor solution and thereafter wash the filtered lignin solids with deionized water.
STAGE 5:
Drying the filtered and washed lignin in an oven.
STAGE 6:
Grinding the oven dried lignin using ball milling method to reduce the lignin particle size.
STAGE 7:
Sieve the lignin powder using different meshes.

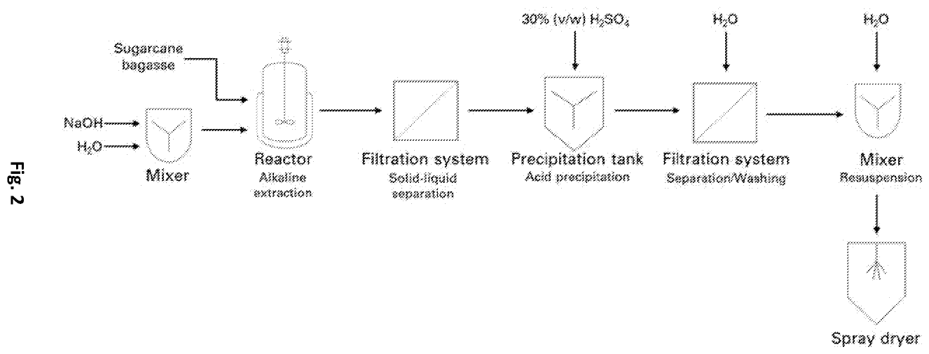

In an embodiment, the lignin of the present disclosure was extracted from SCB and dried using a spray dryer. For the process using the spray dryer, Steps 1-4 are the same as described for the oven (FIG. 2). A brief description of the operating units is as follows:
STAGE 5:
Resuspend the filtered lignin (lignin cake) obtained in the preceding step in deionized water (cake moisture 74-83%).
STAGE 6:
Spray dry the washed resuspended lignin (5% solids (g dry lignin/g solution).

In an embodiment, the alkaline pre-treatment was performed using a mixer and a Parr reactor.

In an embodiment, the solvent preparation was performed as follows:
Prepare the solvent (2 wt % NaOH solution) by adding 30 g of NaOH to 1470 g of deionized water.

In an embodiment, the delignification reaction was performed as follows:
Weight 100 g of SCB for Parr reactor and add 1500 g of solvent (liquid-solid ratio of 15 wt.
Perform the reaction for 0.5 hour (heating and cooling not considered) at 90° C.

In an embodiment, separation of alkaline black liquor (rich in lignin) from solid fraction (rich in cellulose and hemicellulose) was performed as follows:
Separate the black liquor and solid fraction from Stage 1 with a filtration system. On average, 1200 g of liquor (liquor density=1.01±0.01) are obtained.
In this step, on average, 74±2% of the lignin, 5±1% cellulose and 42±7% of hemicellulose initially available in the biomass, respectively, are solubilized in the extracting medium originating from the black liquor.

In an embodiment, precipitation of lignin was performed. Precipitation of lignin present in the alkaline black liquor was achieved by acidification with 30% (v/w) H₂SO₄ (98%).

In an embodiment, precipitation of lignin was performed as follows:
As mentioned in Stage 2, on average, 1200 g of liquor were obtained.
To each 1200 ml of black liquor, add approximately 388 ml of 98% (v/v) H₂SO₄, corresponding to 30% (v/w), at a rate of 100-200 mL/min (the temperature reaches 90° C. at the end).
Afterwards, leave the mixture for one hour at room temperature before entering the next stage: Separation of lignin.

In an embodiment, separation of lignin by filtration or centrifugation, and washing was performed as follows:
Pour the content into a Büchner funnel.
Add 20 L of tap water to the lignin to increase the pH of the lignin to a value of 6.5-7.

In an embodiment, the water content in the lignin is removed to obtain a powder. The water content is removed as follows:
Place the sample obtained in the previous stage (Stage 4) in an oven with forced convection at 40° C. for 16 hours.

In an embodiment, the particle size of the lignin particles of the present disclosure are reduced via ball milling as follows:
Transfer the dried lignin obtained in the previous stage (Stage 5) to the Ball Mill (500 rpm, 1 h).

In an embodiment, the lignin of the present disclosure is fractionated into different particle sizes as follows:
Transfer the dried lignin-rich samples obtained in the previous stage (Stage 5) to the Sieve.

In an embodiment, the lignin of the present disclosure is resuspended in deionized water before being dried by spray dryer as follows:
Mix the wet cake (moisture 72-84%) from stage 4 with deionized water to obtain 5% wt. solids suspension and left under agitation (500 rpm) overnight (16 h). Example: 40.7 g for wet lignin cake (moisture 78%) add 141.4 g of deionized water.

In an embodiment, the lignin of the present disclosure is dried using a spray dryer to obtain a particle size below 40 μm. Preferably, operate the spray dryer (Model Buchi Mini Spray Dryer B-290) using the solid suspension previously prepared (Stage 5). Equipment operating conditions are: 65% aspirator rate, flow height 40-45 mm, pump speed 12%, inlet temperature 160° C.

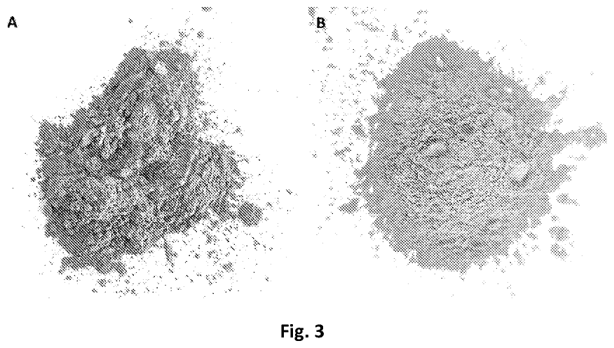

In an embodiment, the characteristics of the lignin of the present disclosure obtained from sugarcane using the two different drying techniques (oven and spray dryer) were evaluated and the results shown in Table 2 below. FIG. 3 shows the colour of each lignin.

TABLE 2
Physicochemical composition of lignin-rich samples obtained
using two drying approaches (oven and spray dryer).

| | Lignin of the present | Lignin of the present |
|---|---|---|
| | disclosure (spray dryer) | disclosure (oven) |
| Raw material | Sugarcane bagasse | Sugarcane bagasse |
| Appearance (Colour, | Light-brown (L* = 60.3, | Light-brown (L* = 48.4, |
| CIELAB space color) | a* = 6.1, b* = 19.1) | a* = 10.0, b* = 20.3) |
| Appearance (Form) | Powder | Powder |
| Odour | Sweet odour with notes of paper wood | Sweet odour with notes of paper |
| | and wooden pencil | wood and wooden pencil |
| Purity (wt %) | 91.9 ± 1.2 | 92.0 ± 0.4 |
| Sugars (wt %) | 2.00 ± 0.12 | 1.86 ± 0.03 |
| Cellulose (wt %) | 1.81 ± 0.13 | 1.73 ± 0.03 |
| Hemicellulose (wt %) | 0.22 ± 0.05 | 0.23 ± 0.03 |
| Ashes (wt %) | 2.2 ± 0.1 | 1.62 ± 0.001 |
| Solubility (mg/mL) | Acetone (4.3 ± 0.1); dimethyl | Acetone (4.0 ± 0.6); dimethyl |
| | sulfoxide (4.4 ± 0.3); methanol | sulfoxide (4.8 ± 1.2); in methanol |
| | (4.2 ± 0.1); ethanol (3.4 ± 0.1); | (4.2 ± 0.3); ethanol (3.7 ± 0.1); |
| | Insoluble in water | Insoluble in water |
| | (0.7 ± 1.1 × 10⁻¹¹). | (0.7 ± 0.2) |
| pH (5% in water) | 4.7 | 5.5 |

In an embodiment, the antioxidant activity of the lignin of the present disclosure was evaluated by Trolox Equivalent Antioxidant Capacity (TEAC) assay, and the results are presented in Table 3. The commercial antioxidant butylated hydroxytoluene (BHT) were also tested as controls.

Table 3 below shows the antioxidant activity by TEAC method of alkaline lignin from sugarcane bagasse obtained using an oven and spray dryer. BHT was used as commercial antioxidant, and commercial lignin was used for comparison purposes (commercial alkali lignin, CAS 8068-05-1, with >95% purity acquired from Sigma-Aldrich) expressed in IC50 values (mg/mL)^{δ}.

TABLE 3
Antioxidant activity by Trolox equivalent antioxidant
capacity (TEAC) method of alkaline lignin from
sugarcane bagasse oven and spray dried.

| Sample | IC50 (mg/mL) |
|---|---|
| Lignin of the present | 0.201 ± 0.006 |
| disclosure oven dried | |
| Lignin of the present | 0.270 ± 0.006 |
| disclosure spray dried | |
| Alkali lignin (CAS 8068-05-1) | 0.146 ± 0.001 |
| Butylated hydroxytoluene | 0.288 ± 0.007 |

^{δ} The IC50 values were given as the mean ± standard deviation of at least three individual determinations each performed in triplicate.

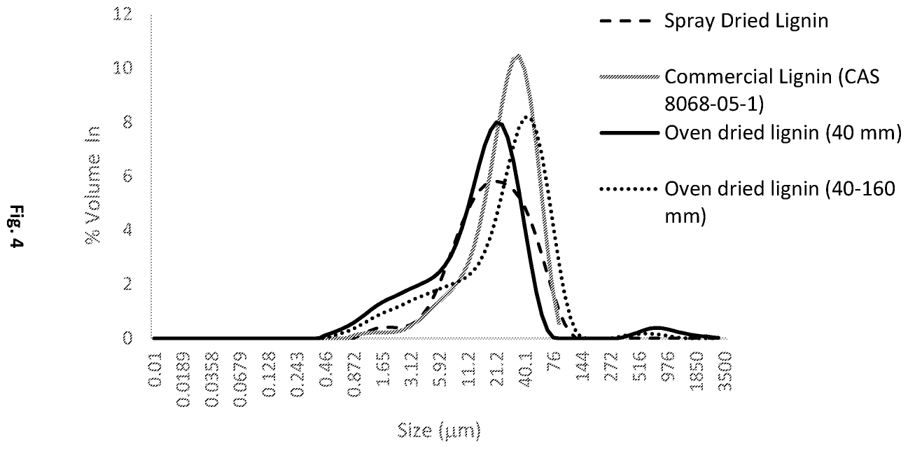

In an embodiment, the size of lignin particles was measured in a MasterSizer Hydro 3000 (Malvern Instruments; Serial number MAL1125347). The solvent employed in the analysis and to disperse the sample was water (refractive index of 1.33). The background was set using water before each analysis. 1 scoop of sample was dispersed in 25 ml of water. The sample was ultrasonicated externally for 5 minutes. The obscuration was set between 5-10%, 60-180 seconds of ultrasounds was applied before each measurement and stirring set to 3500 rpm. It was considered a particle refractive index of 1.64 and absorption index of 0.01. Data was analyzed employing Mie scattering Model and general purpose analysis model. Table 4 shows the standard percentiles D(v, 0.1), D(v,0.5) and D(v,0.9). FIG. 4 shows an example of the particle size distribution obtained by Mastersizer analysis.

TABLE 4
Lignins particle size distribution.

| | Drying | Dv | Dv | Dv |
|---|---|---|---|---|
| Sample | technique | (10) μm | (50) μm | (90) μm |
| Sugarcane | Spray dryer | 5.8-6.8 | 20.0-21.1 | 51.6-57.2 |
| bagasse lignin | Oven | 1.7-2.8 | 15.2-16.8 | 37.0-38.1 |
| of the present | | | | |
| disclosure | | | | |
| Commercial lignin^{§} | — | 9.35-9.45 | 30.1-30.2 | 57.3-57.9 |

^{§}Kraft Lignin, Sigma-Aldrich, CAS 8068-05-1

TABLE 5
Free phenolic hydroxyl and carboxylic groups of lignins.

| | | Free phenolic | Carboxylic |
|---|---|---|---|
| | Drying | hydroxyl groups* | groups** |
| Sample | technique | (mmol/g) | (mmol/g) |
| Sugarcane | Spray dryer | 1.75-1.77 | 1.23 |
| bagasse lignin | Oven | 1.55 | 1.08 |
| of the present | | | |
| disclosure | | | |
| Commercial | — | 1.59-1.84 | 0.89 |
| lignin^{§} | | | |

^{§}Kraft Lignin, Sigma-Aldrich, CAS 8068-05-1
*UV method
**Titration method

In an embodiment, the lignin odour sensorial analysis was performed with a trained descriptive sensory panel and results summarized in Table 6. The sensory evaluation was carried out blindly in order to minimize the perception bias and evaluation results were reported by consensus. Lignin produced herein was firstly presented and evaluated by the sensory panel and afterwards the commercial lignin (reference sample) was presented to the sensory panel since this second sample had a much stronger odour intensity and more marked sensory descriptors that would have influenced the evaluation of the first sample. No odour descriptors listed for the commercial lignin (odour with notes of burnt wood, smoke and spices) were identified in the lignin produced herein. The SCB lignin produced herein had a much lower odour intensity than the commercial sample and the odour was described as mild, sweet with notes of paper, wood and wooden pencil (but not pine wood).

TABLE 6
Sensorial analysis performed with trained panel (6 judges).

| Sample | Odour description |
|---|---|
| Commercial sample^{§} | Odour with notes of burnt wood, |
| | smoke and spices |
| Lignin of the | Mild, sweet odour with notes of paper, wood |
| present disclosure | and wooden pencil (but not pine wood) |
| | Notes of burnt wood, smoke and |
| | spices were absent |

^{§}Kraft Lignin, Sigma-Aldrich, CAS 8068-05-1

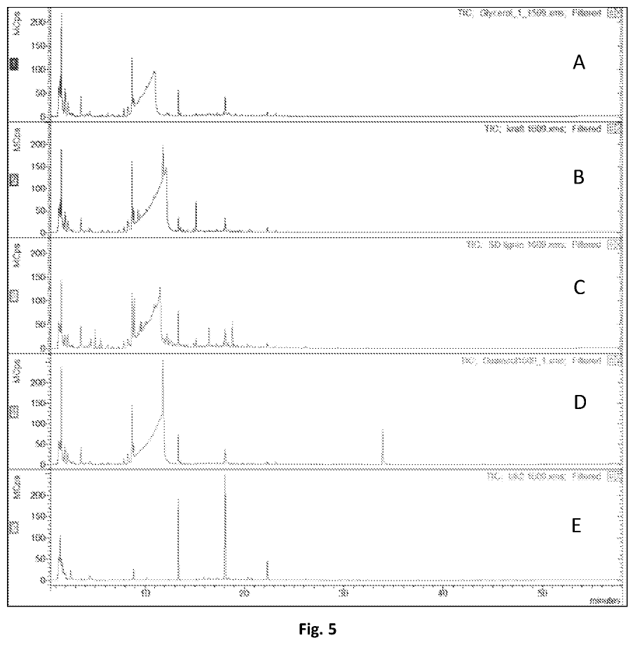

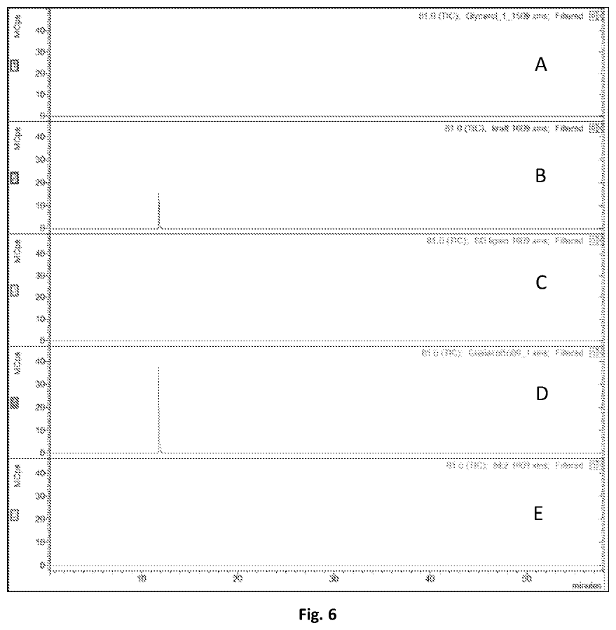

In an embodiment, commercial lignin and lignin produced herein were qualitatively analysed using head space-solid phase microextraction-gas chromatography-mass spectrometry (HS-SPME-GC-MS). Guaiacol is one of the volatile compounds responsible for the strong smoky and woody odour of lignin and was only detected in the commercial lignin sample (FIGS. 5 and 6).

### EXAMPLE

In an embodiment, the potential of lignin as UV blocking agent and as a colorant for use in a cosmetic formulation such as a blemish balm (BB) cream was determined. The in vitro sun protection factor of the cosmetic formulation was assessed. The formulation is original, and all the ingredients were selected based on a clean beauty concept (Table 7).

TABLE 7
Chemical composition of blemish balm (BB) cream formulation
comprising the lignin of the present disclosure.

| Ingredient | wt % |
|---|---|
| Aqua | 63.4 |
| SOLAGUM ™ AX | 0.8 |
| Dermofeel ® PA-12 | 0.1 |
| Glycerin | 5 |
| Phenethylalcohol nat. | 0.9 |
| Tego ® Care PBS 6 MB | 3 |
| Geleol MB | 2 |
| Cetearyl alcohol | 2 |
| Butyrospermum Parkii | 1.5 |
| Caprylic/Capric | 5 |
| Triglyceride | |
| Squalane | 3 |
| Applecare PDS 300 | 0.3 |
| VAS | 7.2 |
| Lignin (particle | 5 |
| size <40 μm) | |
| E-Leen 8 | 0.8 |
| Citric acid 50% (aq, | qs pH 5.0-5.5 |
| w/w)/NaOH 10N | |

In an embodiment, the test product (sample BB cream comprising lignin obtained from the method of the present disclosure) was spread as a thin film on a suitable synthetic substrate and the UV absorbance through this film was measured with a spectrophotometer. The sun protection factor was determined by first irradiating the product with 4 Minimum Erythemal Doses (MEDs) followed by the scanning of the sample from 290 nm to 400 nm. During the scanning the obtained data was accumulated and stored at intervals of 1 nm to determine the monochromatic protection factor (MPF) for each of the selected wavelengths. Then the MPF was used to calculate the SPF value, using solar irradiance and erythemal constants.

In an embodiment, the equipment used for irradiation is a Solar Simulator PV Cell testing 16S-300-002 (Solarlight®, United States of America) serial number #22351. The 16S-300-002 has a 300 watt Xenon arc lamp with a continuous spectrum ranging from 290 to 400 nm. The 16S-300-002 has a vertical beam that directs the light beam to point downward. The spot size is 5.7 cm diameter with one sun output intensity. A suitable warm-up time (at least 10 minutes) is allowed for the UV solar simulator to stabilize before starting exposures to ensure a consistent irradiance over the whole exposure period. Plate exposure is performed at a position within 45.7 cm from the UV beam source.

In an embodiment, a UVB radiometer (SUV Detector PMA2101S-UVS, Solarlight®, United States of America) calibrated against a spectroradiometric measurement of the solar simulator output was used for UV exposure verification.

In an embodiment, DCS 2.0 Dose Controller/Meter (Solarlight®, United States of America), an equipment for SPF testing using the Model 16S Simulator was used to continuously monitor the intensity and the dose being delivered to the PMMA plate. When the dose or time reaches the pre-set value, the shutter automatically closes, terminating the UV irradiation.

In an embodiment, UV absorbance measurements were performed using the SPF-290AS (Solarlight®, United States of America) serial number #290378. The main specifications of the equipment are listed on the following Table 8.

TABLE 8
Specifications of the Spectrophotometer used in the UVB test.

| | |
|---|---|
| Wavelength Range | 290-400 nm |
| Absorbance | 0-3.2 A |
| Wavelength Step | 1 nm, 2 nm, or 5 nm. |
| Interval | User selectable. |
| Lamp | Power stabilized Xenon CW125 W |
| | (operated at 75 W) |

In an embodiment, the thermometer TFA-IP67 was used to measure the temperature that sample experiences under the UV exposure conditions. The thermometer is capable of measuring temperature values ranging from −40° C. to 250° C. with an accuracy of +/−0.5° C. and an uncertainty +/−0.1° C.

In an embodiment, the thermo-hygrometer TFA-TA100 was used to measure the temperature and relative humidity that samples experiences while drying and for absorbance measurements. The thermo-hygrometer is capable of measure temperature values ranging −40° C. to 70° C. with an accuracy of ±0.5° C. for values between 0° C. to 60° C. and for the remaining range ±1° C. The thermo-hygrometer is also capable of measure relative humidity values ranging 0% to 99%. Its accuracy is ±3% and ±5% for values between 35% to 75% and for the remaining range, respectively.

In an embodiment, the PMMA plate dimension preferably has an application area is not less than 16 cm².

The PMMA plate preferably has a molded surface roughness with the following surface parameters within the upper and lower limit values is qualified for use for this in vitro UVB test method (Table 9 below).

TABLE 9
Specifications of PMMA plate used in the UVB test.

| Parameter | Ra | Rv | Rdq | A1 | SSc | Vw |
|---|---|---|---|---|---|---|
| Target value | 4.853 | 13.042 | 11.122 | 239.750 | 0.033 | 1.044E−06 |
| Upper limit | 5.170 | 13.669 | 12.411 | 284.256 | 0.046 | 1.663E−06 |
| Lower limit | 4.535 | 12.414 | 9.833 | 195.244 | 0.020 | 4.248E−07 |

The PMMA plates used as substrate for the sunscreen testing shall pass minimum transmission specifications. This test is performed by applying glycerin or modified glycerin solution to the rough surface of the PMMA plate. Glycerin is spread over the plate with the fingertip and then transmission (between 290 nm to 400 nm) is measured against air (with no plate) as the reference light path.

The limits for the treated plate transmission values are:
290 mn: >60% T; 300 nm: >69% T and 320 nm: >81% T.

In an embodiment, while drying, under UV exposure and for absorbance measurements, the samples of cosmetic formulation comprising lignin are maintained at:
Temperature: 30.0±5.0° C.
Relative humidity: 50.0±10.0° C.

Moreover, the temperature used during the UV exposure and absorbance measurements, is preferably the same used for the drying period. i.e. if UV source exposure conditions are 35° C., then the drying conditions are also at 35° C.; or if the UV source exposure conditions are 25° C., then the drying conditions are also 25° C.

In an embodiment, the SPF value of the cosmetic formulation comprising lignin of the present disclosure was compared to a standard product (Reference sunscreen—comparative example) with a known SPF (16.1±2.4). Reference sunscreen composition is described in Table 10.

TABLE 10
Reference sunscreen (comparative example).

| | | Mass fraction |
|---|---|---|
| Ingredients | | (wt %) |
| Phase 1 | Lanolin | 4.5 |
| | Theobroma cacao | 2.0 |
| | (cocoa seed butter) | |
| | Glyceryl monostearate SE | 3.0 |
| | Stearic acid | 2.0 |
| | Ethylexyldimethyl PABA | 7.0 |
| | (CAS 21245-02-3) | |
| | Benzophenone-3 | 3.0 |
| | (CAS 131-57-7) | |
| Phase 2 | Water | 71.6 |
| | Sorbitol (liquid 70%) | 5.0 |
| | Trieathanolamine (99%) | 1.0 |
| | Methylparaben | 0.3 |
| | Propylparaben | 0.1 |
| Phase 3 | Benzyl alcohol | 0.5 |

The mean SPF and the acceptance limits for the used reference sunscreen formulation are presented below (Table 11).

TABLE 11
Mean SPF of reference sunscreen formulation.

Acceptance limits

| Mean SPF | Lower limit | Upper limit |
|---|---|---|
| 16.1 | 13.7 | 18.5 |

In an embodiment, the comparison analysis was performed. The product application (test product comprising the lignin of the present disclosure (BB cream) or reference product (sunscreen, comparative example)) to the PMMA plate is performed according to the following steps:
1) Remove the protector foil from the polish side of the plate, then clean it with an antistatic cloth to remove any particle or dust.
2) Place the PMMA plate in the balance, record its weigh and tare it.
3) Shake the packaging of the product and apply the quantity corresponding to 32.5±0.3 mg (32.2-32.8 mg) to the roughened surface of the PMMA plate with a pipette, placed directly on the analytical balance, which corresponds to an application of 1.3 mg/cm². Apply as a large number of small droplets of approximate equal volume, distributed evenly over the whole surface of the plate. Record the product amount applied.
4) Spread the product immediately over the whole surface evenly using a saturated gloved finger.
5) Spreading shall be completed in a two-phase process:
   5.1) Firstly, spread the product over the whole area as quickly as possible (less than 30 seconds) using small circular motions with minimal pressure.
   5.2) Secondly, rub the product on the plate surface using alternating horizontal and vertical strokes with increased moderate pressure for 30 seconds.
6) Allow the sample to dry for at least 30 minutes in the dark at the same temperature that will be experienced under the UV exposure conditions.

In an embodiment, the monochromatic protection factor (MPF) is determined for each of the selected wavelengths and is used to calculate the SPF value, using solar irradiance(S) and erythemal constants (E) that are programmed into the software. SPF values are the mean of the 9 measurements at the 9 different locations. The SPF determination for each test product and the reference sunscreen is determined according to the following formula:

$${SPF}_{vitro} = \frac{{\int }_{290 nm}^{400 nm} E ( λ ) * S ( λ )}{{\int }_{290 nm}^{400 nm} E ( λ ) * S ( λ ) / MPF ( λ )} = \frac{{\int }_{290 nm}^{400 nm} E ( λ ) * S ( λ )}{{\int }_{290 nm}^{400 nm} E ( λ ) * S ( λ ) / {10}^{{A}_{λ}}}$$

The mean temperature under UV exposure-before and after the UV irradiation was 30.7±2.0° C. and 30.3±2.3° C., respectively, for the reference sunscreen P2_High SPF reference formula. For test product BB Cream (comprising the lignin of the present disclosure) the mean temperature at sample's distance before and after the UV irradiation was 31.5±0.6° C. and 31.9±0.8° C., respectively. The mean temperature and mean relative humidity immediately before the UV exposure (after drying) are listed on Table 12 and complied with internal specifications for this type of studies (30.0±5.0° C. and 50±10% HR). Moreover, the temperature used during the UV exposure and absorbance measurements was approximately the same used for the drying period.

TABLE 12
Mean room temperatures and relative humidity of
reference sunscreen and each test product immediately
before de UV exposure (after drying).

| | Reference sunscreen | | BB Cream comprising the lignin | |
|---|---|---|---|---|
| | (comparative example) | | of the present disclosure | |
| | T (° C.) | RH (%) | T (° C.) | RH (%) |
| Mean | 25.8 | 41.7 | 26.5 | 41.8 |
| SD | 0.4 | 1.6 | 1.0 | 1.2 |

The mean amount of the reference sunscreen and the test products applied to the PMMA plates are listed on the Table 13 and complied with internal specifications for this study.

TABLE 13
Mean amount of reference sunscreen and
each test product applied to the PMMA.

| | | BB Cream comprising |
|---|---|---|
| | Reference sunscreen | the lignin of the |
| | (comparative example) | present disclosure |
| Amount of product | 32.5 ± 0.2 | 32.5 ± 0.2 |
| applied (mg) | | |

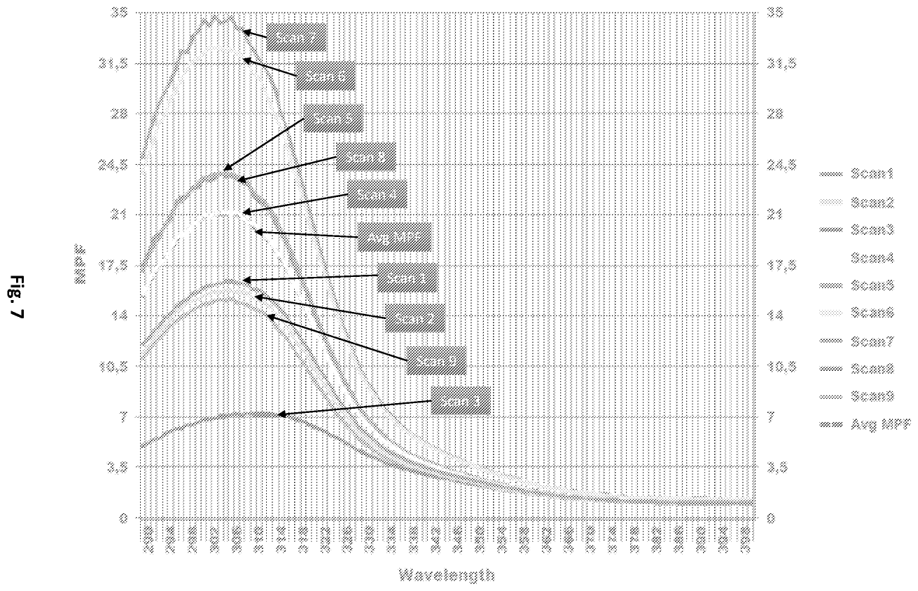

Table 14 shows the mean SPF results of the 9 scans performed for each PMMA plate, obtained for reference sunscreen (comparative example). An example of the MPF graph obtained for each performed scan of one PMMA plate for the reference sunscreen is shown in FIG. 7.

TABLE 14
Mean SPF in vitro value obtained for each reference
sunscreen's (comparative example) PMMA plate.

| PMMA Plate no. | Mean SPF in vitro (9 scans) |
|---|---|
| 1 | 10.63 |
| 2 | 12.86 |
| 3 | 15.25 |
| 4 | 15.40 |
| 5 | 19.51 |
| Mean SPF | 14.73 |
| SD | 3.31 |

According to the obtained results, the SPF in vitro test is valid since the mean SPF value obtained for PMMA plates of reference sunscreen is within the accepted limits, meaning the results obtained for reference product are valid.

In an embodiment, the in vitro SPF test was performed for BB cream comprising the lignin obtain according to the method of the present disclosure.

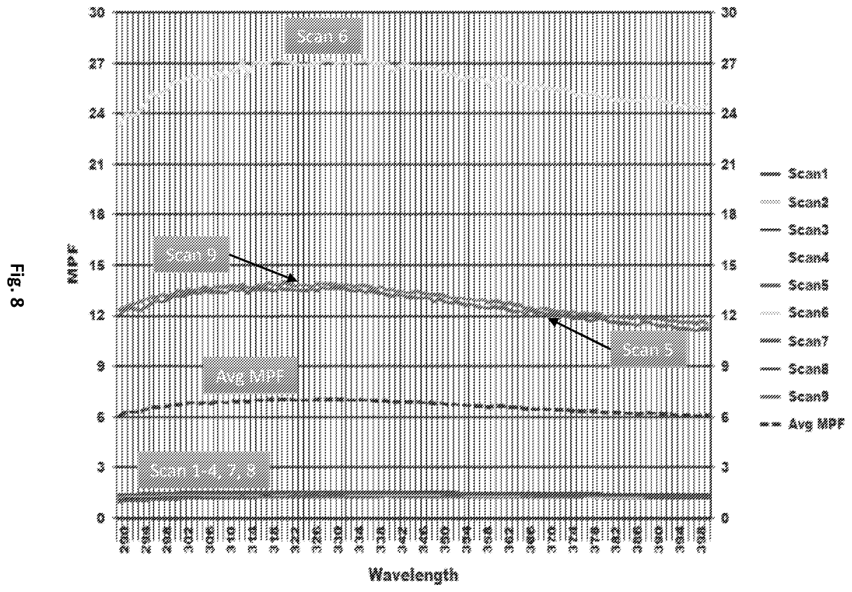

The mean SPF values of the 9 scans performed for each plate where the test product BB Cream was applied are listed on Table 15. An example of the MPF graph obtained for each performed scan of one PMMA plate is shown in FIG. 8.

TABLE 15
Mean SPF in vitro value obtained for each test product's
PMMA plate (BB Cream; Formula of the present disclosure).

| PMMA Plate no. | Mean SPF in vitro (9 scans) |
|---|---|
| 1 | 8.11 |
| 2 | 12.31 |
| 3 | 6.83 |
| 4 | 13.00 |
| 5 | 7.28 |
| MEAN SPF | 9.51 |
| SD | 2.92 |

According to the obtained results for the PMMA plates (Table 14) where the test product was applied, the test product BB Cream (presented a mean in vitro SPF of 9.51±2.92.

In an embodiment, in vivo SPF of sunscreen products provides a basis for the evaluation of sunscreen products for the protection on human skin against erythema induced by solar ultraviolet rays. The SPF test method utilizes a solar lamp simulator of defined and known output to determine the protection provided by sunscreen products on human skin against erythema induced by solar ultraviolet rays.

The test was restricted to the area of the back of selected human subjects. A section of each subject's skin was exposed to ultraviolet light without any protection and another section was exposed to UV light after the application of the sunscreen product under testing. One further section was exposed after application of an SPF reference sunscreen formulation which was used for the validation of the procedure.

In an embodiment to determine the SPF, incremental series of delayed erythemal responses were induced on a number of small sub-sites on the skin. These responses were visually assessed for the presence of redness 20±4 hours after UV radiation, by the judgement of a trained evaluator. The Minimal Erythema Dose (MED) for unprotected skin (MEDu) and the MED obtained after application of a sunscreen product (MED for product protected skin MEDp) were determined on the same subject on the same day. An individual sun protection factor (SPFi) for each subject tested is calculated as the ratio of individual MED on product protected skin divided by the individual MED on unprotected skin MEDp/MEDu. The SPF for the product is the arithmetic mean of all valid SPFi results from each subject in the test.

In an embodiment, a solar simulator (Multiport Simulator model 601-300 W, Solar Light Company) with WG-320 and UG-11 filters, was used as the source of UV irradiation with UV spectral irradiance complying with % RCEE acceptance limits for the UV solar simulator output. Its 6 independently adjustable outputs allow for 6 simultaneously conducted tests. Before and after UV exposure of each test site, the UV irradiance was checked with a calibrated radiometer (PMA2103LLG SUV detector, Solar Light Company). During UV exposure of each test site, the UV-output was dosed using a dose control system (PMA 2100, Solar Light Company, Philadelphia, PA). The test was run on healthy volunteers of both sexes selected according to the inclusion criteria. Volunteers were interviewed by a medically qualified physician to establish their medical status and suitability prior to inclusion into the subjects panel.

In an embodiment, the minimum area for a product application site was 30 cm² and the maximum was 60 cm². The position of the products was random distributed on the back over the whole test group of volunteers in order to reduce systematic error to anatomical differences in skin. The samples were distributed in a quantity equal to 2.00 mg/cm²±2.5%. After product was applied to the skin, the exposure of the test site begins after a waiting period of 15-30 minutes. To aid uniform coverage, droplets (approximately 15 per 30 cm², 30 per 60 cm²) of the product (BB cream) were deposited within the site using a syringe/pipette, then spread over the whole test site using light pressure.

In an embodiment, the method was controlled by the use of one reference sunscreen formulations to verify the test procedure. Reference sunscreen (comparative example) (SPF 16) was used for all the subjects (Table 16).

TABLE 16
SPF in vivo results of Blemish balm cream (ISO 24444: 2019).

| | | MED_{U} | | MED_{P} | | | Results | | |
|---|---|---|---|---|---|---|---|---|---|
| | Skin | | (J/ | | (J/ | | | s | c |
| Subject | ITA | Sec. | m²) | Sec. | m²) | SPF | SPFₘ | (n′) | (n′) |
| 1 | 46.5 | 39 | 241.3 | 352 | 2171.4 | 9.0 | 9.5 | 0.7 | 0.9 |
| 2 | 31.5 | 55 | 452.7 | 499 | 4074.2 | 9.0 | | | |
| 3 | 36.6 | 49 | 350.8 | 445 | 3630.6 | 10.4 | | | |
| 4 | 43.8 | 42 | 296.5 | 376 | 3069.1 | 10.4 | | | |
| 5 | 41.0 | 45 | 419.4 | 402 | 3774.3 | 9.0 | | | |

In an embodiment, the in vitro UVA factor on BB cream was assessed according to the International Organization of Standardization 24444:2019. Specifications are given to enable determination of the spectral absorbance characteristics of UVA protection in a reproducible manner (Table 17).

In an embodiment, the substrate/plate is MOLDED PMMA plates (PolyMethylMethacrylate Plexiglas™) with one side of the substrate roughened. A quantitative of sample was applied and distributed as homogeneous as possible on the PMMA plates. The sample was spotted evenly across the plate surface with a microsyringe. The principle of the analysis is a transmission measurement. The glycerin on the reference substrate serves as a “blank” emulsion (placebo) which contains no light-absorbing or scattering compounds and reduces artificial scattering by the roughened, dry surface much the same as a placebo.

In an embodiment, the determination of the in vitro SPF with the acquisition of the absorbance spectrum of sunscreen product layer previously spread on the plate and reading at the spectrophotometer Labsphere 2000S. Transmission measurements are performed on at least 10 areas for each plate and at least 4 plates are tested, for each sample. The interested wavelength is in the range 290-400 nm. Mathematical adjustment of the curve with all the transmittance values that constitute the absorbance curve from 290-400 nm (absorbing region of UVA-UVB rays), that were multiply for the same coefficient C, to let correspond the obtained SPF in vitro result with the in vivo one.

In an embodiment, the UVA protection (UVAPF 0) was calculated using the normalised spectrum (320-400 nm); the “D” dose was evaluated from the UVAPF0 value multiplied for 1.2 Joules/cm².

In an embodiment, the exposure time of the sample to the SUN TEST was estimated from the UVAPF0 value; the acquisition of the absorbance spectrum of the sunscreen product layer previously spread on the plate, but after the SUN TEST exposure (second absorbance spectrum) (Table 18).

TABLE 17
UVA results for BB cream.

| ISO in vitro UVA | |
|---|---|
| protection test method | Results |
| Product name | BB cream |
| SPF measured in vivo | 9 |
| Adjustment coefficient | 1.5 |
| for SPFO = SPF (Co) | |
| Spectro Analyser | Labsphere 2000 S |
| Applied amount of product per area | 1.3 mg/cm² |
| Plate manufacturer/Lot number | PMMA plated MOLDED no00361 |
| Solar simulator for UV exposure | Sun test CPS+ |
| Raw UVA irradiance | 59.6 |

TABLE 18
In vitro UVA PF results (ISO 24443: 2012).

| | Mean | Std dev | IC 95% | IC 95% |
|---|---|---|---|---|
| UVA PF 0 (STATIC) | 4.86 | 0.18 | 0.28 | |
| ISO in vitro UVA PF | 3.41 | 0.21 | 0.33 | 9.7% |
| (UVAPF(Dx)) | | | | |

Critical wavelength value = 378 ± 0.5 nm

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A lignin comprising a light-brown colour, wherein the colour is at least L*=45, a*=6, b*=17 scale measured by the CIELAB system;
a particle size inferior to 70 μm;
wherein the volatile organic compounds profile is absent of guaiacol;
wherein lignin has a sweet and woody odour, preferably with notes of paper wood and wooden pencil;
wherein the lignin particle size is inferior to 60 μm; and
wherein the degree of purity of the lignin is at least 85%.

2. (canceled)

3. The lignin according to claim 1 wherein the lignin is obtainable from sugarcane bagasse.

4. The lignin according to claim 1 wherein the lignin is obtained by spray dryer.

5. The lignin according to claim 1, wherein the lignin comprises an in vitro ultraviolet light absorbance ranging from 2-12, measured at the global solar irradiance in the UV wavelength ranging from 290-400 nm.

6. The lignin according to claim 1 wherein the degree of purity of the lignin is at least 95%.

7. A composition comprising the lignin described in claim 1 and a suitable cosmetic base.

8. (canceled)

9. A method of use of the lignin according to claim 1 as a cosmetic additive, as a pigment, as an ultraviolet blocking agent, as an antioxidant, as a skin care agent, as a preventer and/or retardant skin ageing.

10. (canceled)

11. (canceled)

12. A method for treating or preventing sunburn or solar skin diseases in a subject, the method comprising administering the lignin of claim 1 to the subject.

13. (canceled)

14. (canceled)

15. The composition as described in claim 7 wherein the suitable cosmetic base is selected from a list consisting of emulsifier, humectant, thickener, stabilizer, preservative, chelating agent, emollient, pH adjuster, or mixtures thereof.

16. The composition as described in claim 7 wherein:
the emulsifier is selected from a group consisting of: Polyglyceryl-6 distearate; glyceryl stearate, cetearyl alcohol, polysorbate-20, bee wax, and mixtures thereof;
the humectant is selected from a group consisting of: Glycerin, propylene glycol, xanthan gum, salicylic acid, hyaluronic acid, and mixtures thereof; and
the thickener is selected from a group consisting of: Xanthan gum, hydroxyethylcellulose, sclerotium gum, acacia Senegal gum, carbomers, and mixtures thereof.

17. (canceled)

18. (canceled)

19. The composition as described claim 7 wherein the stabilizer is selected from a group consisting of: Acacia Senegal gum, xanthan gum, carbomers, or sodium gluconate, triethanolamine, and mixtures thereof.

20. The composition as described in claim 7 wherein the preservative is selected from a group consisting of: Phenethyl alcohol, benzoic acid, sorbic acid, salicylic acid alcohol, pentylene glycol, and mixtures thereof.

21. The composition as described in claim 7 wherein the chelating agent is selected from a group consisting of: Sodium phytate, citric acid, EDTA, sodium oxalate, and mixtures thereof.

22. The composition as described in claim 7 wherein the emollient is selected from a group consisting of: Butyrospermum parkii butter, caprylic/capric triglyceride, bee wax, squalane, cetyl alcohol, dimethicone, lanolin, and mixtures thereof.

23. The composition as described in claim 7 wherein the pH adjuster is selected from a group consisting of: Citric acid, aqua/sodium hydroxide, ascorbic acid, acetic acid, magnesium hydroxide, and mixtures thereof.

24. The composition as described in claim 7 wherein the composition comprises 1-10 wt % of lignin and 90-99 wt % % of base; preferably 3-7wt % of lignin and 93-97 wt % of base; more preferably 4-5 wt % of lignin and 95-96 wt % of base.

25. The composition as described in claim 7 wherein the composition is a cosmetic composition.

26. (canceled)

27. The composition as described in claim 7 wherein the cosmetic composition is in the form of a topical composition.

28. The composition according to claim 27 wherein the topical composition is a gel, a lotion, a liquid, a balm, a cream or a serum, a spray.

29. The composition according to claim 27 wherein the topical composition is a blemish balm or a colour correcting cream.

30. (canceled)

31. (canceled)

32. (canceled)

33. (canceled)

34. (canceled)

35. (canceled)

36. (canceled)

37. (canceled)
